(19) United States
(12) Patent Application Publication
(10) Pub. No.: US 2023/0343425 A1
(43) Pub. Date: Oct. 26, 2023

(54) METHODS AND NON-TRANSITORY COMPUTER STORAGE MEDIA OF EXTRACTING LINGUISTIC PATTERNS AND SUMMARIZING PATHOLOGY REPORT

(71) Applicant: TAIPEI MEDICAL UNIVERSITY, Taipei City (TW)

(72) Inventors: Cheng-Yu CHEN, TAIPEI CITY (TW); Yung-Chun CHANG, TAIPEI CITY (TW); Shih-Hsin HSIAO, TAIPEI CITY (TW)

(21) Appl. No.: 17/726,559

(22) Filed: Apr. 22, 2022

(51) Int. Cl. G16H 15/00; G06F 40/295; G06F 40/166

(52) U.S. Cl. CPC G16H 15/00; G06F 40/295; G06F 40/166; G06F 40/30

(57) ABSTRACT

Disclosed are methods and the non-transitory computer storage media of extracting linguistic patterns and summarizing a pathology report thereof. The present disclosure provides a method of extracting key linguistic patterns from a pathology report. The method comprises: determining a confidence degree and a support degree between a linguistic term and a next linguistic term based on co-occurrences of the linguistic term and the next linguistic term; generating a set of candidate linguistic terms; generating a first set of linguistic patterns through performing random walks on the set of candidate linguistic terms; and determining the key linguistic patterns through removing redundant linguistic patterns from the first set of linguistic patterns.

### FIELD OF THE INVENTION

The present disclosure relates to a method of processing a pathology report. In particular, the present disclosure relates to a method of extracting key linguistic patterns from a pathology report, a method of summarizing a pathology report, and the non-transitory computer storage media thereof. The present disclosure further relates to a method of determining similarities between pathology reports.

### BACKGROUND

A pathology report of a patient includes a large amount of information, especially for cancer patients, and such pathology report includes a substantial amount of miscellaneous and tedious information. The surgeon and the physician in charge may spend much time to understand a patient's situation, but computers may be helpful in reducing the amount of time wasted and thus may increase overall efficiency.

### SUMMARY OF THE INVENTION

The subject disclosure can analyze a pathology report. A pathology report may contain the diagnosis determined by examining cells and tissues under a microscope. The report may be for a lung cancer patient. Important messages can be summarized from a miscellaneous and tedious pathology report. Such messages may include six categories of features: basic description in pathology, tumor features, histological description, immunohistochemistry (IHC) information, a genetic testing result, and a pathological TNM (tumor, node and metastasis) stage. The present disclosure can further summarize multiple pathology reports of one patient. The present disclosure can further provide a function of searching among data of a large amount of patients, and the search result can be a reference for the surgeon and the physician.

An embodiment of the present disclosure provides a method of extracting key linguistic patterns from a pathology report. The method comprises: determining a confidence degree and a support degree between a linguistic term and a next linguistic term based on co-occurrences of the linguistic term and the next linguistic term; generating a set of candidate linguistic terms; generating a first set of linguistic patterns through performing random walks on the set of candidate linguistic terms; and determining the key linguistic patterns through removing redundant linguistic patterns from the first set of linguistic patterns. The linguistic term occurs prior to the next linguistic term in the pathology report. The confidence degree between a candidate linguistic term and a corresponding next candidate linguistic term is equal to or greater than a confidence threshold. The support degree between the candidate linguistic term and the corresponding next candidate linguistic term is equal to or greater than a support threshold.

Another embodiment of the present disclosure provides a method of summarizing a pathology report. The method comprises acquiring a plurality of pathological features from the pathology report based on key linguistic patterns. The key linguistic patterns are generated according to any one of the methods or operations of the present disclosure.

A further embodiment of the present disclosure provides a non-transitory computer storage medium. The non-transitory computer storage medium has program instructions stored thereon. Upon execution of the program instructions by a processor, the program instructions cause performance of a set of operations according to any one of the methods of the present disclosure.

### BRIEF DESCRIPTION OF THE DRAWINGS

In order to describe the manner in which advantages and features of the present disclosure can be obtained, a description of the present disclosure is rendered by reference to specific embodiments thereof, which are illustrated in the appended drawings. These drawings depict only example embodiments of the present disclosure and are not therefore to be considered limiting of its scope.

FIG. 1 illustrates a pathological semantic correlation graph according to some embodiments of the present disclosure.

FIG. 2 illustrates a flow diagram of a method of extracting key linguistic patterns according to some embodiments of the present disclosure.

FIG. 3 is a section of a pathology report according to some embodiments of the present disclosure.

FIG. 4 is a section of a pathology report according to some embodiments of the present disclosure.

FIG. 5 is a part of a pathology report according to some embodiments of the present disclosure.

FIG. 6 illustrates a schematic diagram of data representations of pathology reports according to some embodiments of the present disclosure.

FIG. 7 illustrates a schematic diagram showing a computer system in accordance with some embodiments of the present disclosure.

### DETAILED DESCRIPTION

The following disclosure provides many different embodiments, or examples, for implementing different features of the provided subject matter. Specific examples of operations, components, and arrangements are described below to simplify the present disclosure. These are, of course, merely examples and are not intended to be limiting. For example, a first operation performed before or after a second operation in the description may include embodiments in which the first and second operations are performed together, and may also include embodiments in which additional operations may be performed between the first and second operations. For example, the formation of a first feature over, on or in a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Time relative terms, such as “prior to,” “before,” “posterior to,” “after” and the like, may be used herein for ease of description to describe one operation or feature's relationship to another operation(s) or feature(s) as illustrated in the figures. The time relative terms are intended to encompass different sequences of the operations depicted in the figures. Further, spatially relative terms, such as “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly. Relative terms for connections, such as “connect,” “connected,” “connection,” “couple,” “coupled,” “in communication,” and the like, may be used herein for ease of description to describe an operational connection, coupling, or linking one between two elements or features. The relative terms for connections are intended to encompass different connections, coupling, or linking of the devices or components. The devices or components may be directly or indirectly connected, coupled, or linked to one another through, for example, another set of components. The devices or components may be wired and/or wirelessly connected, coupled, or linked with each other.

As used herein, the singular terms “a,” “an,” and “the” may include plural referents unless the context clearly indicates otherwise. For example, reference to a device may include multiple devices unless the context clearly indicates otherwise. The terms “comprising” and “including” may indicate the existences of the described features, integers, steps, operations, elements, and/or components, but may not exclude the existences of combinations of one or more of the features, integers, steps, operations, elements, and/or components. The term “and/or” may include any or all combinations of one or more listed items.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified.

The nature and use of the embodiments are discussed in detail as follows. It should be appreciated, however, that the present disclosure provides many applicable inventive concepts that can be embodied in a wide variety of specific contexts. The specific embodiments discussed are merely illustrative of specific ways to embody and use the disclosure, without limiting the scope thereof.

To summarize important pathological features from a pathology report (e.g., a pathology report of lung cancer), the present disclosure provides a method named PR2Sum (Pathology Report to Summary). In some embodiments of the present disclosure, 50 important pathological features among six categories are selected, filtered, or defined from a pathology report. 50 exemplary pathological features are listed in Table 1. The six categories may include: the basic description, the finding (of tumor(s)), the histology (information of tumor(s)), the IHC information, the genetic testing (result), and the TNM stage. In further embodiments, the data representation of report(s) of a patient may be represented by the 50 pathological features shown in Table 1.

TABLE 1

| Category | Pathological Features |
|---|---|
| Basic | organ, Bx-site, sampling method, diagnosis |
| Description | |
| Finding | greatest dimension, tumor size, |
| | closest margin, lymphovascular |
| | invasion, VPI, tumor focality |
| Histology | histology type, histology grade |
| IHC | CK7, TTF-1, Napsin A, CK20, P40, CDX2, P63, P16, |
| | cytokeratin(AE1/AE3), Vimentin, PAX-8, CD56, |
| | chromogranin-A, synaptophysin, GATA3, |
| | P53, S100, Ki67, EBER |
| Genetic | EGFR, ALK, ROS1, BRAF, MET, KRAS, ERBB2, |
| testing | PIK3CA, NRAS, MEK1, NTRK, RET, PDL1 |
| TNM stage | version, pT, pN, pM, pStage, N info |

With machine learning algorithms (or deep learning algorithms), various different combinations of features must be continuously tried to increase the performance during training the model. The processes of generating various different combinations of features may be called feature engineering. The feature engineering for the machine learning algorithms is expensive. For example, feature engineering for the machine learning algorithms would spend much time to generate various feature combinations and test the combinations. However, the feature combinations obtained from such a high-cost model may only be applicable to the current research field. For example, an entirely new set of feature combinations should be generated for the pathology reports associated with another disease. Once the research field or disease is changed, the feature engineering should be started over for a new set of feature combinations, and the model should be trained again with the new set of different feature combinations. It is difficult for the constructed or trained machine learning models to achieve the effect of knowledge sharing. Additionally, known machine learning algorithms for similar issues are not interpretable because such machine learning algorithms only generates a lot of uninterpretable parameters and probabilities.

Human knowledge can be accumulated. In human thinking, the situation/condition of an article or a problem stated in a language can be narrowed down through some important linguistic terms. For example, when a clinician interprets a pathology report, if the terms “immunohistochemically,” “immunoreactive,” and “Napsin-A” concurrently appear in the report, he/she will spontaneously view the corresponding sentence as relating to the immunohistochemical staining reaction of Napsin-A because said three terms have a strong correlation. That is, humans can read an article by skimming it. If the terms in a pathology report can meet the knowledge framework of a clinician, the clinician can understand what pathological feature is described in a sentence or paragraph.

Human's perception of a topic is obtained through the identification of important entities or related contents to scope out possible candidates. For example, when highly correlated words like “Immunohistochemically” and “Napsin-A” appear in a sentence simultaneously, it is natural to conclude that this is more likely to be a sentence describing the patient's response to Napsin-A immunohistochemical staining. The present disclosure may be similar to what humans do when they skim a pathology report to capture its main idea. Moreover, the acquired knowledge from different topics can be accumulated and adopted to recognize other new topics.

In light of this, the method of the present disclosure imitates the perceptual behavior of human's comprehension, which is a highly automatic approach that learns linguistic patterns that characterize the domain of lung cancer from the raw text in pathology reports. One of the main advantage of the present disclosure is the high precision and capability for knowledge accumulation. Confronted with a new domain, the knowledge can be further extended by adding new rules to adapt to unknown information.

Different from machine learning algorithms, the present disclosure provide a novel method for natural-language understanding. Regarding the acquirement of pathological features, the present disclosure simulates the behaviors of a clinician while reading a pathology report (e.g., a pathology report of a lung cancer patient). The present disclosure can quickly narrow down the situation/condition of an article or a problem through realizing important points or linguistic terms. The reasons include the strong correlations between the gist of the article (or the problem) and the adjacent terms. Thus, the gist or the problem can be identified naturally. Therefore, the methods or algorithms provided in the present disclosure can be interpretable.

In addition to acquiring the important features of a pathology report, the present disclosure also emphasizes flexible comparisons which cannot be carried out by ossified regular expressions. Thus, the present disclosure has a higher degree of freedom during pathological linguistic pattern matching.

The present disclosure provides a pathological linguistic pattern generation algorithm. The pathological linguistic pattern generation algorithm can be used for lung cancer patients. In some embodiments, the pathological linguistic pattern generation algorithm can be used patients of various cancers, including, but limited to, prostate cancer, colorectal cancer, stomach cancer, breast cancer, colorectal cancer, and cervical cancer. In some embodiments of the present disclosure, the linguistic patterns for identifying pathological feature of lung cancer can be generated based on the pathological reports of 500 lung cancer patients.

In the present disclosure, the processes of generating pathological linguistic patterns for lung cancer can be viewed as a problem of frequent pattern mining. A pathological semantic correlation graph for lung cancer can be constructed based on the co-occurrences of the terms in the pathology reports. The pathological semantic correlation graph can describe the strength semantic correlation between different terms.

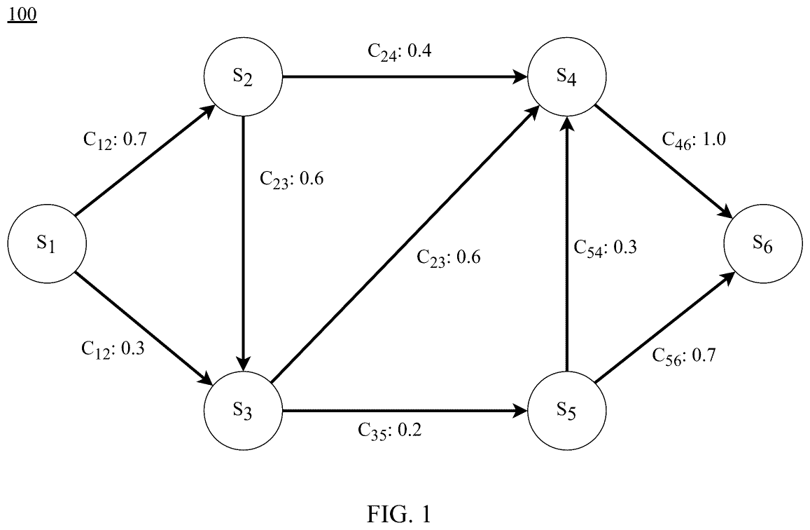

FIG. 1 illustrates a pathological semantic correlation graph 100 according to some embodiments of the present disclosure. The pathological semantic correlation graph 100 can be constructed based on the co-occurrences of the terms in one or more pathology reports of one or more lung cancer patients. In some embodiments, the pathological semantic correlation graph 100 is constructed based on the terms appearing in multiple pathology reports of multiple patients. After the occurrence frequencies of each term appearing in the pathology reports and the co-occurrence times between different terms appearing in the pathology reports are calculated, the pathological semantic correlation graph 100 can be constructed.

Since the pathological linguistic patterns (e.g., for lung cancer) to be generated may be an ordered directed graph, the present disclosure constructs a semantic correlation graph with association rules.

Each vertex in FIG. 1 may indicate different terms. For example, terms (or phrases) S₁ and S₂ are represented as different vertexes. Each edge in FIG. 1 is constructed based on the co-occurred terms (or phrases). For example, the co-occurrence C₁₂ of the terms S₁ and S₂ are represented as the edge between the corresponding vertexes, in which the term S₁ occurs prior to the term S₂. In other words, co-occurrence C₁₂ indicates the co-occurrence of the term S₁ occurs prior to the next term S₂. The value of the co-occurrence Cᵢⱼ indicates the confidence degree for the terms Sᵢ and Sⱼ. The value of the co-occurrence Cᵢⱼ indicates a conditional probability, which a measure of the probability of the term Sⱼ occurring, given that the term Sᵢ has already occurred. In some embodiments, the term S₁, co-occurring with the term S₂, may be a superordinate term of the term S₂. The term S₁, co-occurring with the term S₂, may be a subordinate term of the term S₂. The confidence degree for the terms Sᵢ and Sⱼ is defined as Equation (1).

$$Confidence ( {S}_{i} ⇒ {S}_{j} ) = P ( {S}_{j} \vert {S}_{i} ) = \frac{P ( {S}_{j} ⋂ {S}_{i} )}{P ( {S}_{i} )} \qquad Equation ( 1 )$$

The support degree may be defined as “the number of samples of the true response that lie in that class.” In present disclosure, the support degree of the term Sᵢ may indicate the frequency of the occurrences of the term Sᵢ. In some embodiments, the support degree of the term Sᵢ may indicate the number of occurrences of the term Sᵢ. The support degree of the term Sᵢ and the next term of Sⱼ may indicate the frequency of the co-occurrences of the term Sᵢ and the next term of Sⱼ. The support degree of the term Sᵢ and the next term Sⱼ may indicate the number of co-occurrences of the term Sᵢ and the next term Sⱼ. The relation between a confidence degree and the corresponding support degree may be defined as Equation (2).

$$Confidence ( {S}_{i} ⇒ {S}_{j} ) = \frac{P ( {S}_{j} ⋂ {S}_{i} )}{P ( {S}_{i} )} = \frac{Support ( {S}_{i} ⋃ {S}_{j} )}{Support ( {S}_{i} )} \qquad Equation ( 2 )$$

In order to make the generated linguistic patterns have discrimination with regard to the pathological features, the terms having higher frequency are retained in some embodiments of the present disclosure. The pathological semantic correlation graph for lung cancer can be constructed according to the number of co-occurrences of the retained frequent terms. In some embodiments of the present disclosure, the minimum support degree is set to 10, and the minimum confidence degree is set to 0.3 so as to avoid generating linguistic patterns that are too short.

After the pathological semantic correlation graph for lung cancer is constructed, the terms with higher frequency and better discrimination are strung together based on random walks. The linguistic patterns of a pathological feature can be generated based on the terms which are strung together.

A pathological semantic correlation graph for lung cancer may be defined as G=(V,E), in which |V|=p, |E|=k. V indicates the set of vertexes (e.g., the set of vertexes for terms S₁ to S₆ shown in FIG. 1), E indicates the set of edges (e.g., the set of edges for the co-occurrences Cᵢⱼ shown in FIG. 1), p indicate the number of elements in the set V, and k indicates the number of elements in the set E. A process of random walks can be performed on the graph G. The process of random walks may consist of a series of random paths. The value of Cᵢⱼ indicates the probability of the movement from the term Sᵢ to the term Sⱼ. For one term Sₙ, the set of all the adjacent terms may be represented as N(Sₙ). For one term Sₙ, the sum of the probabilities of the movements from term Sₙ to each term of the set N(Sₙ) meets with Equation (3).

$$∀ {S}_{n} {\sum }_{{S}_{m} ∈ 𝒩 ( {S}_{n} )} {C}_{n m} = 1 \qquad Equation ( 3 )$$

Through applying a random walk process to the pathological semantic correlation graph G, the obtained probability matrix Pr complies with Equation (4). Xₖ indicates the k-th step of the random walk process. Therefore, a series of randomly generated vertexes is a Markov Chain.

$$\begin{matrix}Pr = [ {X}_{t + 1} = {S}_{m} \vert {X}_{t} = {S}_{n} , {X}_{t - 1} = {S}_{k} , … , {X}_{0} = {S}_{i} ] \\ = [ {X}_{t + 1} = {S}_{m} \vert {X}_{t} = {S}_{n} ]\end{matrix} \qquad Equation ( 4 )$$

According to the research results of Dr. L Lovász, the cover time (CT) on a graph can be represented as Equation (5).

∀Sₙ,CT_{S}_{ₙ}≤4|k|² Equation (5)

Therefore, through applying the theory of random walks on the pathological semantic correlation graph for lung cancer, the present disclosure can search possible linguistic frequent patterns. In addition to avoiding the loss of combinations with lower probability, the present disclosure can generate the linguistic patterns for different pathological features.

The present disclosure describes the correlations between each frequent term through a pathological semantic correlation graph for lung cancer. Then, through applying the process of random walks on the pathological semantic correlation graph, the terms which frequently occur in the pathology reports are strung together, and the linguistic patterns for pathological features can be generated.

However, some redundant linguistic patterns may be generated through the theory of random walks, and some further integrations may be necessary. In some embodiments, the present disclosure removes a linguistic pattern which is completely included in another linguistic pattern and thereby tries to retain the linguistic patterns (or linguistic frames) with longer length and better cover rate. For example, the linguistic patterns (or linguistic frames) of “S₁→S₂→S₃” and “S₁→S₄→S₂→S₃→S₅→S₆” are generated, and the former pattern would be retained because the former pattern includes the latter pattern. In other words, if a second linguistic pattern is a subset of a first linguistic pattern, the second linguistic pattern would be removed, and the first linguistic pattern would be retained. Furthermore, if a first linguistic pattern dominates a second linguistic pattern, the second linguistic pattern would be removed, and the first linguistic pattern would be retained. After removing the specific linguistic patterns, each of the remaining linguistic patterns may be a set of unordered terms/phrases (Sᵢ) or a set of ordered terms/phrases (Sᵢ).

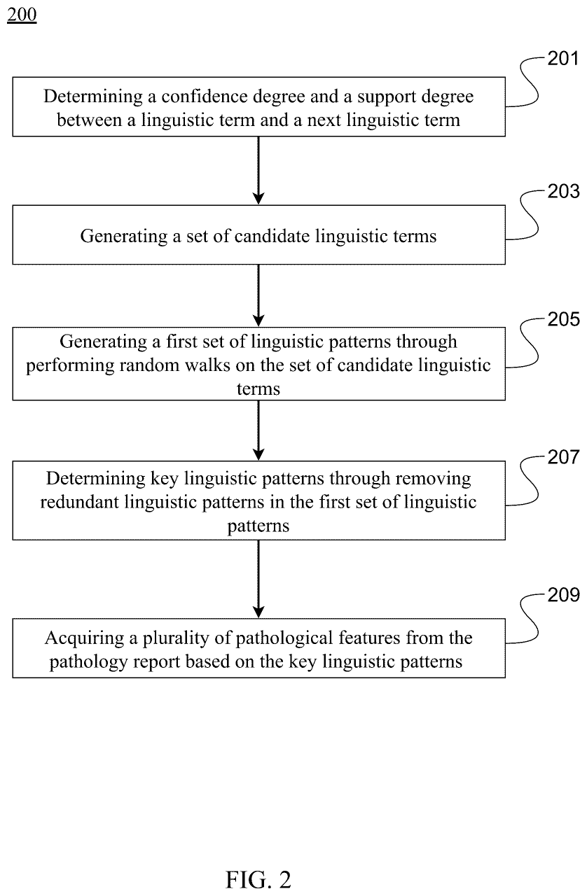

FIG. 2 a flow diagram of a method 200 according to some embodiments of the present disclosure. The method 200 can extract key linguistic patterns from one or more pathology reports of one or more patients. The method 200 includes operation 201. In the operation 201, a confidence degree and a support degree between a linguistic term and a next linguistic term may be determined based on the co-occurrences of the linguistic term and the next linguistic term. In the one or more pathology report, the linguistic term may occur prior to the next linguistic term.

The method 200 includes operation 203. In the operation 203, a set of candidate linguistic terms (or phrases) may be generated or selected from the one or more pathology reports. In the set of candidate linguistic terms, the confidence degree value between one candidate linguistic term and the corresponding next candidate linguistic term is equal to or greater than a confidence threshold. In the set of candidate linguistic terms, the support value degree between one candidate linguistic term and the corresponding next candidate linguistic term is equal to or greater than a support threshold.

The method 200 includes operation 205. In the operation 205, a first set of linguistic patterns may be generated or selected from the set of candidate linguistic terms. The first set of linguistic patterns may be generated or selected through performing random walks on the set of candidate linguistic terms.

The method 200 includes operation 207. In the operation 207, the key linguistic patterns may be generated or selected from the first set of linguistic patterns. The key linguistic patterns may be generated or selected through removing redundant linguistic patterns from the first set of linguistic patterns. Each of the key linguistic patterns may be a set of unordered terms/phrases. Each of the key linguistic patterns may be a set of ordered terms/phrases.

The method 200 includes operation 209. In the operation 209, a plurality of pathological features may be acquired from one or more pathology reports based on key linguistic patterns. The plurality of pathological features may include the exemplary 50 pathological features listed in Table 1. The acquired, extracted, or summarized pathological features forming a pathology can allow clinicians to quickly understand the situation or condition of the corresponding patient.

In some embodiments of the present disclosure, the support degree between a linguistic term and the next linguistic term may be generated or calculated based on the number of co-occurrences of the linguistic term and the next linguistic term. The confidence degree value between a linguistic term and the next linguistic term may be generated or calculated based on a probability of occurrence of the next linguistic term under the case in which the linguistic term occurs.

In some embodiments of the present disclosure, the confidence threshold may be set to 0.3, and the support threshold may be set to 10. The confidence threshold may be selected within the range of 0.2 to 0.5. The support threshold may be selected within the range of 7 to 12.

In some embodiments of the present disclosure, a first linguistic pattern is removed when a second linguistic pattern is a subset of the first linguistic pattern. That is, a first linguistic pattern is removed if the first linguistic pattern includes a second linguistic pattern.

In some embodiments of the present disclosure, the confidence degree between a linguistic term and the next linguistic term is irrelevant to a second confidence degree value between a previous linguistic term and the linguistic term. The previous linguistic term occurs prior to the linguistic term in the pathology report, and the linguistic term occurs prior to the next linguistic term.

In some embodiments of the present disclosure, 50 linguistic patterns for the important pathological features of lung cancer are generated through the pathological linguistic pattern generation algorithm. After verifying with clinicians, the method of acquiring the pathological features for the six categories, including: the basic description, the finding of tumor(s), the histological description of tumor(s), the IHC information, the genetic testing result, and the TNM stage, are described as follows.

A pathology report includes information categorized in the basic description. Such basic description may be provided in the SOAP (Subjective, Objective, Assessment, and Plan) section. “Subjective” may indicate subjective data, including the chief complaint, symptom, medical history, drug allergy history, adverse drug reaction history, and medication history expressed by the patient. “Objective” may indicate objective data, including vital signs, physical exam results, lab test results, and medical imaging results of the patient. “Assessment” may include the impression/diagnosis, the patient's conditions, the disease conditions, and the analysis and assessment of the therapy. “Plan” may include approaches to diagnosis (lab tests), approaches to therapy (medications, procedures, operations, etc.), and approaches to healthcare education.

The content of the SOAP section can be separated or divided into multiple parts by commas (i.e., “,”). In some embodiments, when the number of parts separated by commas in the SOAP section is less than 4, the content of the SOAP section may be the test results. For lung cancer patients, when the number of parts separated by commas in the SOAP section is greater than or equal to 4, the content of the SOAP section may be the lung description.

Table 2 shows four exemplary SOAP sections. For each of Cases 1, 2, and 4 in Table 2, the number of parts is determined to be less than 4. For each of Cases 1, 2, and 4 in Table 2, the content of the SOAP section is determined to be related to the test results. For Case 3 in Table 2, the number of parts is determined to be greater than 4.

TABLE 2

| Case | SOAP Section |
|---|---|
| 1 | The ALK expression is negative. |
| 2 | Rearrangement of ROS1 gene is NOT detected. |
| 3 | Lung, lower lobe, right, bronchoscopic biopsy, adenocarcinoma |
| 4 | EGFR exon 18 mutation: No mutation detected |
| | EGFR exon 19 mutation: No mutation detected |
| | EGFR exon 20 mutation: Insertions in exon 20 (EX20Ins) |
| | EGFR exon 21 mutation: No mutation detected |

In some embodiments, when the number of parts separated by commas in the SOAP section is equal to 4, these four parts would be related to “organ,” “location,” “sampling method,” and “diagnosis,” in sequence. That is, the first part would be related to one or more organs, the second part would be related to one or more locations, the third part would be related to one or more sampling methods, and the four part would be related to one or more diagnoses.

In some embodiments, when the number of parts separated by commas in the SOAP section is greater than 4, some parts may be merged. For example, the linguistic patterns generated for the basic description indicate some key linguistic patterns for the part related to “organ” and some key linguistic patterns for the part related to “sampling method.” One or more parts related to “organ” can be located or identified through some key linguistic patterns. One or more parts related to “sampling method” can be located or identified through some key linguistic patterns. For lung cancer patients, exemplary key linguistic patterns for locating parts related to “organ” and “sampling method” are listed in Table 3. After locating the one or more parts related to “organ” and “sampling method,” one or more parts related to “location” can be located between those related to “organ” and “sampling method.” For example, if the first part and fifth part are related to “organ” and “sampling method,” respectively, the second to fourth parts would be determined to be related to “location.” One or more parts posterior to those related to “sampling method” would be determined to be related to “diagnosis.” For example, if the SOAP section is divided into seven parts and if the fifth part is related to “sampling method,” the sixth and seventh parts would be determined to be related to “diagnosis.” In some preferred embodiments, only one part posterior to those related to “sampling method” would be determined to be related to “diagnosis.”

TABLE 3

| Part | Key Linguistic Patterns |
|---|---|
| Organ | lung, Lymph node, Brain, Skin |
| Sampling | biopsy, VATS, EBUS |
| Method | |

For Case 3 in Table 2, the content of the SOAP section is “Lung, lower lobe, right, bronchoscopic biopsy, adenocarcinoma.” For Case 3, the number of parts is determined to be greater than 4. Based on the key linguistic patterns in Table 3, the part “Lung” in Case 3 would be determined to be related to “organ,” and the part “bronchoscopic biopsy” in Case 3 would be determined to be related to “sampling method.” In Case 3, the parts “lower lobe” and “right” would be determined to be related to “location” because they are located between the parts related to “organ” and “sampling method.” In Case 3, the part “adenocarcinoma” would be determined to be related to “diagnosis” because it is posterior to the part related to “sampling method.” For Case 3 in Table 2, the parts related to “organ,” “location,” “sampling method,” and “diagnosis” are marked with grey color.

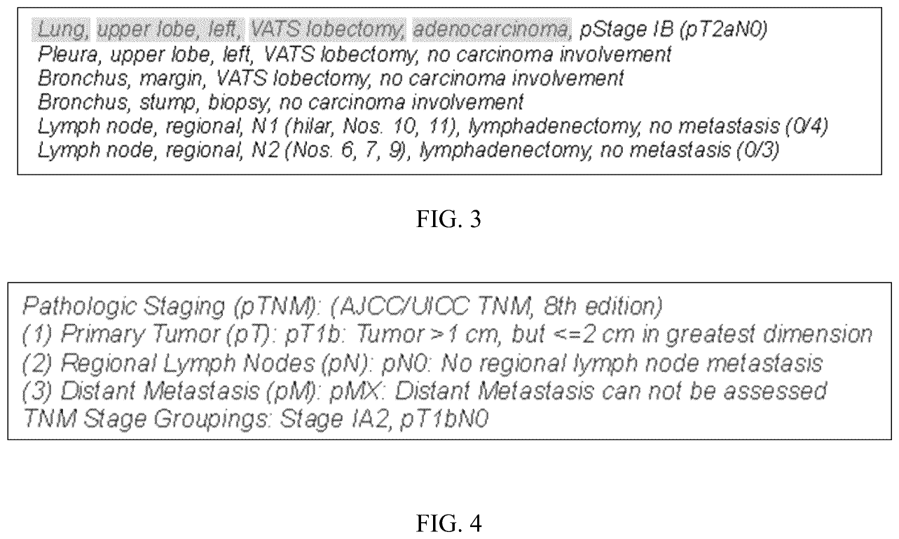

FIG. 3 illustrates a section of a pathology report according to some embodiments of the present disclosure. FIG. 3 shows the SOAP section of a pathology report. In the case of FIG. 3, the SOAP section includes 6 sentences. The number of parts separated by commas of each sentence in FIG. 3 would be determined to be greater than 4. According to the present disclosure, these six sentences having more than 4 parts would be selected as candidates for further processing. For each sentence in FIG. 3, it would be determined which part(s) is related to “organ” based on one or more key linguistic patterns for the part related to “organ” listed in Table 3. For each sentence in FIG. 3, it would be determined which part(s) is related to “sampling method” based on one or more key linguistic patterns for the part related to “organ” listed in Table 3. After the parts related to “organ” and “sampling method” are determined, the part(s) between the parts related to “organ” and “sampling method” would be determined to be related to “location.” One part posterior to that related to “sampling method” would be determined to be related to “diagnosis.” For the case of FIG. 3, the output basic description would be “organ: Lung/location: upper lobe, left/sampling method: VATS lobectomy/diagnosis: adenocarcinoma.” In the case of FIG. 3, the parts related to “organ,” “location,” “sampling method,” and “diagnosis” are marked with grey color.

The one or more key linguistic patterns for the part related to “organ” listed in Table 3 may be selected or generated for a lung cancer patient. The one or more key linguistic patterns for the part related to “organ” listed in Table 3 may include terms other than “lung.” The reason is that the metastasis or invasion from the lung cancer may occur in other organs. Thus, the one or more key linguistic patterns for the part related to “organ” listed in Table 3 may include terms like “Lymph node,” “Brain,” and “Skin.”

In some embodiments of the present disclosure, the content in the parts related to “location” and “diagnosis” may be further standardized for output. For example, the content in the part(s) related to “location” may include various types of descriptions. The content in the part(s) related to “location” can be further standardized with at least one of “left upper lobe,” “left lower lobe,” “right upper lobe,” “right middle lobe,” or “right lower lobe.” If the tumor invades other organs, the content in the part(s) related to “location” may be further standardized with the at least one of “pleural,” “Bone,” “Brain,” or “Skin.” The content in the part(s) related to “diagnosis” may also include various types of descriptions. The content in the part(s) related to “diagnosis” can be further standardized with at least one of “Adenocarcinoma,” “Non-small Cell Carcinoma,” “Squamous Cell Carcinoma,” or “Large Cell Carcinoma.”

A pathology report includes information categorized in the finding of tumor(s). According to some embodiments of the present disclosure, the linguistic patterns generated for the finding of tumor(s) are associated with volume. The linguistic patterns for the finding of tumor(s) may be a linguistic pattern of volume. The linguistic patterns for the finding of tumor(s) may include multiple numbers, multiple multiplication symbols, and a unit. For example, the linguistic patterns for the finding may include three numbers, two multiplication symbols, and a unit. The first multiplication symbol may be between the first and the second numbers. The second multiplication symbol may be between the second and the third numbers. The unit may be posterior to the third number. The unit may be a unit of length (e.g., “cm” or “mm”) or a unit of volume.

According to some embodiments of the present disclosure, one or more candidate segments may be selected based on the linguistic patterns generated for the finding of tumor(s). If the context of the one or more candidate segments includes one or more terms of the key linguistic patterns listed in Table 4, the associated one or more volume values would be determined as the information of tumor size. Furthermore, the greatest value of the values indicating a volume would be determined as the value for the greatest dimension. For example, “50 mm×35 mm×20 mm” includes three values to indicate a volume, and the greatest value, “50,” soul de determine as the value for the greatest dimension.

TABLE 4
Key Linguistic Patterns for Tumor Size

on cut, firm tumor, tumor measuring

In addition to the finding of tumor(s), a pathology report includes information categorized in the histological information of tumor(s). The information of the finding of tumor(s) and the histological information of tumor(s) may be observed through microscopes in a lab. The information observed through microscopes may be described in different items of a pathology report. The items associated microscopic information may be described in the “microscopic evaluation” section of a pathology report.

In some embodiments of the present disclosure, the microscopic evaluation section may be located or identified based on the term “microscopic evaluation.” The information of an item in the microscopic evaluation section may be described after a colon (i.e., “:”). In some embodiments of the present disclosure, the information described in each item can be acquired through locating the corresponding colon (i.e., “:”). For example, after locating the microscopic evaluation section, a given item can be located by searching the corresponding item name, the corresponding colon of the given item is then located, and the information located posterior to the colon of the given item will be acquired as the information of the item. The microscopic evaluation section may include at least one item of “tumor focality,” “histology type,” “histology grade,” “lymphovascular invasion,” “visceral pleura invasion,” and “closest margin.” The items of “tumor focality,” “lymphovascular invasion,” “visceral pleura invasion,” and “closest margin” may be categorized in the finding of tumor(s). The items of “histology type” and “histology grade” may be categorized in the histological information of tumor(s).

A pathology report includes information categorized in the IHC information. According to some embodiments of the present disclosure, the linguistic patterns generated for the IHC information indicates that the terms listed in Table 5, which may be from one or more key linguistic patterns, can be regarded as an initial term of the IHC section.

TABLE 5
Key Linguistic Patterns for an Initial Term of the IHC Section

Immunohistochemical, Immunohistochemically, Immunohistochemical,
Immunohistochemically, Immunostudy, IHC, Immunostains,
Immunohistochemistry, Immunostudy, immunoreactive, shows
adenocarcinoma composed

After locating or identifying the IHC section by the initial term, the information of the IHC section can be further acquired. The target item in the IHC section can be located or identified based on one or more key linguistic patterns listed in Table 6.

After target items in the IHC section are located or identified, for each target item, a first modifier can be located or identified prior to the target item, and a second modifier can be located or identified posterior to the target item. For each target item, a first distance between the first modifier and the target item and a second distance between the second modifier and the target item can be calculated. For each target item, if the first distance is smaller than the second distance, the first modifier would be determined as the modifier for the target item; if the second distance is smaller than the first distance, the second modifier would be determined as the modifier for the target item. The first modifier and the second modifier may be “positive” or “negative.” When the first modifier and the second modifier are identical, it would be unnecessary to select or determine which modifier is used to modify the target term. Thus, the calculations of the first and second distances may be unnecessary. Furthermore, the comparison between the first and second distances may be unnecessary.

TABLE 6
Key Linguistic Patterns for Target Items of the IHC Section

CK7, TTF-1, Napsin A, CK20, P40, CDX2,
P63, P16, cytokeratin(AE1/AE3),
Vimentin, PAX-8, CD56, chromogranin-A, synaptophysin, GATA3, P53,
S100, Ki67, EBER

A pathology report includes information categorized in the genetic testing. The information for the genetic testing may include the testing of the immune checkpoint inhibitor (e.g., PDL1 inhibitor), the genetic testing of the epidermal growth factor receptor (EGFR), and other genetic molecular testing.

In some embodiments of the present disclosure, the linguistic patterns generated for testing of the immune checkpoint inhibitor indicate some terms related to PDL1 testing kits. One or more exemplary key linguistic patterns related to PDL1 testing kits are listed in Table 7.

TABLE 7
Key Linguistic Patterns for PDL1 Testing

22C3, 28-8, SP142, SP263

Searches on the entire pathology report based on one or more terms of one or more key linguistic patterns listed in Table 7 are conducted. Whether the PDL1 testing was performed can be determined based on such searches. For example, if searches on the entire pathology report hit one or more terms of the key linguistic patterns listed in Table 7, it may be determined that the PDL1 testing was performed. If the PDL1 testing is performed, the information of the items associated with the PDL1 testing would be further acquired. The items associated with the PDL1 testing include: tumor proportion score (TPS), combined positive score (CPS), tumor cell (TC), and immune cells (IC).

The key linguistic patterns listed in Table 7 can be used to locate or identify the PDL1 testing part. The items associated with the PDL1 testing may be provided in the PDL1 testing part. The information of an item associated with the PDL1 testing may be described after a colon (i.e., “:”). In some embodiments of the present disclosure, the information described in each item can be acquired through locating the corresponding colon (i.e., “:”). For example, after locating the PDL1 testing part, a given item can be located by searching the item name; the corresponding colon of the given item can be then located, and the information located posterior to the colon of the given item will be acquired as the information of the item.

In some embodiments of the present disclosure, the linguistic patterns generated for testing of the EGFR indicate one or more terms, e.g., the term “EGFR.” Searches on the entire pathology report based on the term “EGFR” may be conducted. Whether the EGFR testing was performed can be determined based on such searches. For example, if searches on the entire pathology report hit the term “EGFR” or other similar terms, it may be determined that the EGFR testing was performed. If the EGFR testing is performed, the information about the mutations in exon 18, exon 19, exon 20, and exon 21 of the EGFR would be further acquired.

The term “EGFR” can be used to locate or identify the EGFR testing part. The information about the mutations in exon 18, exon 19, exon 20, and exon 21 of the EGFR may be provided in the EGFR testing part. In some embodiments, whether mutations are in exon 18, exon 19, exon 20, or exon 21 may be determined based on searches for the terms of “18,” “19,” “20,” and “21.” For example, if searches hit the term “18” in the EGFR testing part, it would be determined that a mutation is in exon 18. In some other embodiments, whether a mutation is at position 790 of exon 20 may be determined based on searches for the term “T790M.” For example, if searches hit the term “T790M” in the EGFR testing part, it would be determined that a mutation is at position 790 of exon 20.

In some embodiments of the present disclosure, the linguistic patterns generated for other genetic molecular testing indicate some key linguistic patterns listed in Table 8. Searches on the entire pathology report based on the terms of the key linguistic patterns listed in Table 8 may be conducted. Whether some specific genetic molecular testing was performed can be determined based on such searches. For example, if searches on the entire pathology report hit one or more terms of the one or more key linguistic patterns listed in Table 8, it may be determined that the corresponding genetic molecular testing was performed.

TABLE 8
Key Linguistic Patterns for Genetic Molecular Testing

ALK, ROS1, BRAF, MET, KRAS, ERBB2,
PIK3CA, NRAS, MEK1, NTRK, RET

The key linguistic patterns listed in Table 8 can be used to locate or identify specific genetic molecular testing parts. After one genetic molecular testing part is located or identified, the information related to the result can be further acquired. For example, if the context of one genetic molecular testing part includes the term “positive,” it indicates that a mutation may occur in the corresponding gene. If the context of one genetic molecular testing part includes the term “negative,” it indicates that a mutation may not occur in the corresponding gene.

In some embodiments, after one genetic molecular testing part is located or identified, a first modifier can be located or identified prior to the corresponding key linguistic pattern, and a second modifier can be located or identified posterior to the corresponding key linguistic pattern. For the located genetic molecular testing part, a first distance between the first modifier and the corresponding key linguistic pattern and a second distance between the second modifier and the corresponding key linguistic pattern can be calculated. If the first distance is smaller than the second distance, the first modifier would be determined as the modifier for the located genetic molecular testing part; if the second distance is smaller than the first distance, the second modifier would be determined as the modifier for the located genetic molecular testing part. The first modifier and the second modifier may be “positive” or “negative.”

A pathology report includes information categorized in the pathological TNM stage. In some embodiments of the present disclosure, the linguistic patterns generated for the pathological TNM stage indicate some terms. One or more exemplary key linguistic patterns for the pathological TNM stage are listed in Table 9.

TABLE 9
Key Linguistic Patterns for Pathological TNM Stage Section

pathologic staging, pTNM, AJCC

Searches on the entire pathology report based on one or more terms of one or more key linguistic patterns listed in Table 9 are conducted. The terms of one or more key linguistic patterns listed in Table 9 can be used to locate or identify the section of pathological TNM stage. For example, the pathological TNM stage section may be the “Pathological Staging (pTNM)” section.

FIG. 4 illustrates a pathological TNM stage section of a pathology report according to some embodiments of the present disclosure. Searches on the entire pathology report based on the terms of one ore more key linguistic patterns listed in Table 9 may be conducted. For example, if searches on the entire pathology report hit one or more terms of one or more key linguistic patterns listed in Table 9, the pathological TNM stage section can be located or identified. FIG. 4 shows an example in which the terms “pathologic staging,” “pTNM,” and “AJCC” are located in the beginning of the pathological TNM stage section. The staging information of the items in the pathological TNM stage section would be further acquired. The items in the pathological TNM stage section include: pT (i.e., Primary Tumor), pN (i.e., Regional Lymph Nodes), and pM (Distant Metastasis).

In some embodiments, after locating the pathological TNM stage section, the version number provided after the term “AJCC” (i.e., American Joint Committee on Cancer) would be acquired. The staging information may be provided posterior to or below the version number of AJCC. For example, FIG. shows that the items “Primary (pT),” “Regional Lymph Nodes (pN),” and “Distant Metastasis (pM)” and the corresponding information are provided below the version number of AJCC.

The associated linguistic pattern indicates that the staging result may be followed by the name of a given item. For example, the staging result for the item “Primary Tumor (pT)” can be found after the name of the item (e.g., “Primary Tumor” or “pT”). FIG. 4 shows that the staging result “pT1B” of the item “Primary (pT)” is provided after the item name.

The staging information of an item in the pathological TNM stage section may be described after a colon (i.e., “:”). In some embodiments of the present disclosure, the information described in each item can be acquired through locating the corresponding colon (i.e., “:”). For example, after locating the pathological TNM stage section, a given item can be located by searching the item name; the corresponding colon of the given item can be then located, and the information located posterior to the colon of the given item will be acquired as the information of the item. FIG. 4 shows that the staging result “pN0” of the item “Regional Lymph Nodes (pN)” is provided after the corresponding colon.

In some embodiments, the pathological TNM stage section may further include the item “TNM Stage Groupings.” FIG. 4 shows a pathological TNM stage section including the item “TNM Stage Groupings.” The information of the item “TNM Stage Groupings” indicates a TNM stage based on the information of the items “Primary (pT),” “Regional Lymph Nodes (pN),” and “Distant Metastasis (pM).”

The associated linguistic pattern indicates that the TNM staging result may be followed by the name of the item “TNM Stage Groupings.” FIG. 4 shows that the TNM staging result “Stage IA2” of the item “TNM Stage Groupings” is provided after the item name.

The TNM staging information of the item “TNM Stage Groupings” may be described after a colon (i.e., “:”). In some embodiments of the present disclosure, the TNM staging information described in each item can be acquired through locating the corresponding colon (i.e., “:”). For example, after locating the item by searching the item name “TNM Stage Groupings,” the corresponding colon of the item can be then located, and the TNM staging information located posterior to the colon of the item will be acquired as the TNM staging information. FIG. 4 shows that the TNM staging result “Stage IA2” of the item “TNM Stage Groupings” is provided after the corresponding colon.

In some embodiments, the pathological TNM stage section may not include the item “TNM Stage Groupings.” The present disclosure can acquire the TNM staging information based on the staging information of the pT item, the pN item, and the pM item. In some embodiments the TNM staging information can be acquired through looking up Table 10. Table 10 is based on the 8th edition of the TNM staging system of AJCC/UICC (International Union for Cancer Control). In Table 10, T1, T2, T3, T4 and the corresponding subcategories are stages for “Primary Tumor (pT)”; N0, N1, N2, and N3 are stages for “Regional Lymph Nodes (pN)”; and M1 and the corresponding subcategories are stages for “Distant Metastasis (pM).” In Table 10, IA1, IA2, IA3, IB, IIA, IIB, IIIA, IIIB, IIIC, IVA, and IVB are stages for “TNM Stage Groupings.”

TABLE 10

| T/M | Subcategory | N0 | N1 | N2 | N3 |
|---|---|---|---|---|---|
| T1 | T1a | IA1 | IIB | IIIA | IIIB |
| | T1b | IA2 | IIB | IIIA | IIIB |
| | T1c | IA3 | IIB | IIIA | IIIB |
| T2 | T2a | IB | IIB | IIIA | IIIB |
| | T2b | IIA | IIB | IIIA | IIIB |
| T3 | T3 | IIB | IIIA | IIIB | IIIC |
| T4 | T4 | IIIA | IIIA | IIIB | IIIC |
| M1 | M1a | IVA | IVA | IVA | IVA |
| | M1b | IVA | IVA | IVA | IVA |
| | M1c | IVB | IVB | IVB | IVB |

In some embodiments of the present disclosure, a field of “N info” may be generated to verify or supplement the information of the item “Regional Lymph Nodes (pN).” In some embodiments, a pathology report may provide the examined lymph nodes and the associated information. The present disclosure can further verify the staging information of “Regional Lymph Nodes (pN)” based on the examined lymph nodes and the associated information.

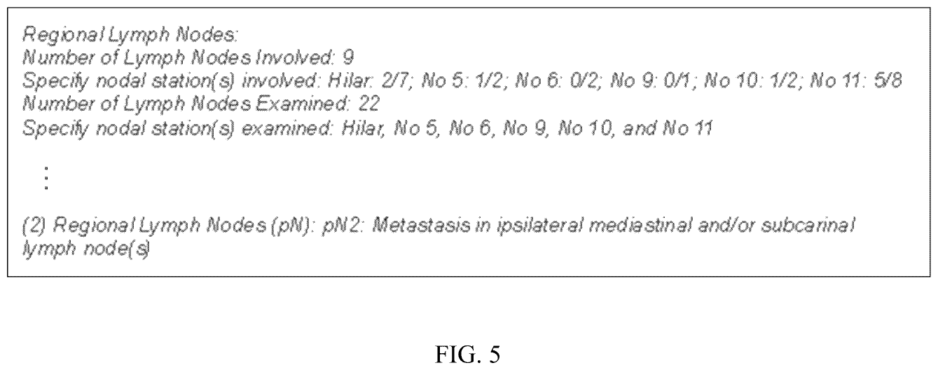

FIG. 5 illustrates a part of a pathology report according to some embodiments of the present disclosure. In FIG. 5, the examined lymph nodes and the associated information are provided. In FIG. 5, the third line indicates that: seven lymph nodes are examined at the “Hilar” position, in which 2 lymph nodes are involved; five lymph nodes are examined at the “No 5” position, in which 1 lymph node is involved; two lymph nodes are examined at the “No 6” position, in which no lymph nodes are involved; one lymph node is examined at the “No 9” position, in which no lymph node is involved; two lymph nodes are examined at the “No 10” position, in which one lymph node is involved; and eight lymph nodes are examined at the “No 11” position, in which five lymph nodes are involved.

The present disclosure would determine the pN stage based on the involved or invaded lymph nodes. In the pathology report shown in FIG. 5, nine lymph nodes are involved or invaded. Based on a staging method for clinicians, the staging results for different positions in the pathology report of FIG. 5 can be obtained. The stage for the “Hilar” position is N2 because 2 of 7 lymph nodes are involved. The stage for the “No 5” position is N2 because 1 of 2 lymph nodes is involved. The stage for the “No 10” position is N1 because 1 of 2 lymph nodes is involved. The stage for the “No 11” position is N1 because 5 of 8 lymph nodes are involved. The pN stage would be determined as the largest stage among the staging results for different positions. For the pathology report shown in FIG. 5, the pN stage would be determined as “N2” because “N2” is the largest stage among the stages of the “Hilar,” “No 5,” “No10,” and “No 11” positions. In the pathology report of FIG. 5, the item “Regional Lymph Nodes (pN)” also provides the stage “N2.”

To verify the performance of the methods provided in the present disclosure, 849 pathology reports from 203 lung cancer patients are provided to a program according to some embodiments of the present disclosure. The accuracy of the exemplary 50 pathological features of lung cancer is provide in Table 11. For an entire pathology report, the methods of the present disclosure can provide an overall accuracy of 86.69%.

TABLE 11

| Basic | organ | Bx-site | sampling method | diagnosis | |
|---|---|---|---|---|---|
| Description | 98.8% | 89.5% | 97.9% | 93.6% | |
| Finding | greatest | tumor size | closest margin | | |
| | dimension | | | | |
| | 98.5% | 98.2% | 99.5% | | |
| | lymphovascular | VPI | tumor focality | | |
| | invasion | | | | |
| | 99.4% | 99.8% | 99.8% | | |
| Histology | histology type, | histology | | | |
| | | grade | | | |
| | 97.9% | 100% | | | |
| IHC | CK7 | TTF-1 | Napsin A | CK20 | P40 |
| | 98.3% | 95.4% | 97.1% | 99.8% | 98.6% |
| | CDX2 | P63 | P16 | Cytokeratin | Vimentin |
| | | | | (AE1/AE3) | |
| | 100% | 100% | 99.8% | 99.8% | 100% |
| | PAX-8 | CD56 | chromogranin-A | synaptophysin | GATA3 |
| | 100% | 100% | 99.8% | 100% | 99.7% |
| | P53 | S100 | Ki67 | EBER | |
| | 100% | 99.8% | 99.2% | 100% | |
| Genetic | EGFR | ALK | ROS1 | BRAF | MET |
| testing | 100% | 100% | 99.2% | 100% | 100% |
| | KRAS | ERBB2 | PIK3CA | NRAS | MEK1 |
| | 100% | 100% | 100% | 100% | 100% |
| | NTRK | RET | PDL1 | | |
| | 100% | 100% | 98.8% | | |
| TNM stage | version | pT | pN | pM | pStage |
| | 100% | 98.3% | 99.7% | 99.8% | 94.8% |
| | N info | | | | |
| | 100% | | | | |

In some embodiments of the present disclosure, if a patient has multiple pathology reports, these pathology reports would be summarized, combined, and divided into “an initial diagnosis report” and “a newest diagnosis report.” The time stamps of the initial diagnosis and the newest diagnosis should be determined. For example, after arranging the multiple pathology reports in time sequence, the time stamps of the initial diagnosis and the newest diagnosis may be the dates of the first non-puncture pathology report and the latest non-puncture pathology report, respectively.

According to the experience of clinicians, information for genetic testing and IHC stain testing may not appear in the first non-puncture pathology report and the latest non-puncture pathology report. Genetic testing and IHC stain testing may be performed one month after the first non-puncture pathology report and the latest non-puncture pathology report. Thus, the content of the pathology reports within a month from the time stamp of the first non-puncture pathology report or the latest non-puncture pathology reports should be monitored or reserved. The information about the basic description, the finding of the tumor, and the histology in the first non-puncture pathology report or the latest non-puncture pathology report may be reserved. The information about the genetic testing and the IHC stain testing may be summarized and combined with those about the basic description, the finding of the tumor, and the histology. In some embodiments, the information in the latest non-puncture pathology report and the information about the genetic testing and the IHC stain testing in the subsequent pathology report(s) may be for a patient who has been treated via surgery or therapy.

According to some embodiments of the present disclosure, each of the multiple pathology reports of one patient would be summarized based on the exemplary 50 pathological features. Upon summarization, each of the multiple pathology reports of one patient may be represented in terms of the exemplary pathological features. The data representation of each of the multiple pathology reports of one patient can be represented in terms of the exemplary 50 pathological features. The multiple pathology reports which have been represented in terms of the exemplary 50 pathological features can be combined based on the sequence of the time stamps.

The data representation of a pathology report of one patient can be represented in terms of the exemplary 50 pathological features. In some embodiments, if the data of a given pathological feature is descriptive data, the descriptive data can be represented with a sentence embedding method. In some embodiments of the present disclosure, the sentence embedding method may be trained based on pathology reports, and may be 300-dimensional. Sentence embedding is the collective name for a set of techniques in natural language processing (NLP), where sentences are mapped to vectors of real numbers. Sentence embedding is a technique for representing a descriptive feature, in which the text or the paragraph is mapped or projected to a high dimensional space and the meaning of the descriptive feature is represented with a high dimensional space or a high dimensional vector.

The data representation of a pathology report of one patient can be represented in terms of the exemplary 50 pathological features. Thus, pathology reports can be comparable to one another. However, the importance of each pathological feature may be different from each other. Therefore, in some embodiments of the present disclosure, different pathological features may be weighted with different weight values. The weight values for the exemplary 50 pathological features are provided in Table 12. According to some embodiments of the present disclosure, a data representation of a pathology report may be represented in terms of the exemplary 50 pathological features. Furthermore, the data representation of a multiple pathology report can be normalized in terms of the weighted 50 pathological features, in which the weighted 50 pathological features may be weighted with the weight values provided in Table 12.

TABLE 12

| Basic | organ | Bx-site | sampling method | diagnosis | |
|---|---|---|---|---|---|
| Description | 100 | 5 | 100 | 100 | |
| Finding | greatest | tumor size | closest margin | | |
| | dimension | | | | |
| | 1 | 1 | 20 | | |
| | lymphovascular | VPI | tumor focality | | |
| | invasion | | | | |
| | 20 | 20 | 5 | | |
| Histology | histology type, | histology | | | |
| | | grade | | | |
| | 100 | 5 | | | |
| IHC | CK7 | TTF-1 | Napsin A | CK20 | P40 |
| | 5 | 20 | 5 | 5 | 5 |
| | CDX2 | P63 | P16 | Cytokeratin | Vimentin |
| | | | | (AE1/AE3) | |
| | 5 | 20 | 20 | 5 | 5 |
| | PAX-8 | CD56 | chromogranin-A | synaptophysin | GATA3 |
| | 1 | 5 | 5 | 5 | 5 |
| | P53 | S100 | Ki67 | EBER | |
| | 40 | 5 | 5 | 5 | |
| Genetic | EGFR | ALK | ROS1 | BRAF | MET |
| testing | 100 | 100 | 100 | 100 | 100 |
| | KRAS | ERBB2 | PIK3CA | NRAS | MEK1 |
| | 100 | 100 | 100 | 100 | 100 |
| | NTRK | RET | PDL1 | | |
| | 100 | 100 | 50 | | |
| TNM stage | version | pT | pN | pM | pStage |
| | 5 | 5 | 5 | 5 | 100 |
| | N info | | | | |
| | 5 | | | | |

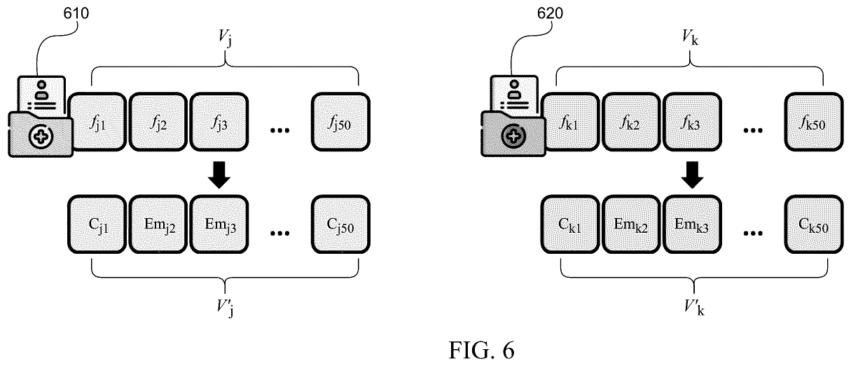

FIG. 6 illustrates a diagram of data representations of pathology reports 610 and 620 according to some embodiments of the present disclosure. The pathology reports 610 and 620 may belong to the same patient or different patients. The pathology report 610 may be represented as a pathology feature vector Vⱼ. The pathology feature vector Vⱼ may include several features summarized or extracted from the pathology report 610. The features of the pathology feature vector Vⱼ may be summarized or extracted from the pathology report 610 through one or more methods or algorithms of the present disclosure. In FIG. 6, the pathology feature vector Vⱼ includes features fⱼ₁ to fⱼ₅₀. The features fⱼ₁ to fⱼ₅₀ may be numeric data or descriptive data corresponding to the 50 features listed in the Table 1, Table 11, or Table 12. In the embodiments of FIG. 6, the features fⱼ₁ and fⱼ₅₀ are numeric data, and the features fⱼ₂ and fⱼ₃ are descriptive data.

The pathology report 620 may be represented as a pathology feature vector Vₖ. The pathology feature vector Vₖ may include several features summarized or extracted from the pathology report 620. The features of the pathology feature vector Vₖ may be summarized or extracted from the pathology report 620 through one or more methods or algorithms of the present disclosure. In FIG. 6, the pathology feature vector Vₖ includes features fₖ₁ to fₖ₅₀. The features fₖ₁ to fₖ₅₀ may be numeric data or descriptive data corresponding to the features listed in the Table 1, Table 11, or Table 12. In the embodiments of FIG. 6, the features fₖ₁ and fₖ₅₀ are numeric data, and the features fₖ₂ and fₖ₃ are descriptive data.

For each of the features with numeric data (e.g., each of the features fⱼ₁, fⱼ₅₀, fₖ₁, and fₖ₅₀ in FIG. 6), the numeric data can be further transformed into a category type data. For example, the features fⱼ₁, fⱼ₅₀, fₖ₁, and fₖ₅₀ in FIG. 6 are transformed into categories Cⱼ₁, Cⱼ₅₀, Cₖ₁, and Cₖ₅₀, respectively. In some embodiments, when the numeric data of feature fⱼ₁ is lower than a given threshold, the value of category Cⱼ₁ may be 0; when the numeric data of feature fⱼ₁ is greater than the given threshold, the value of category Cⱼ₁ may be 1.

For each of the features with descriptive data (e.g., each of the features fⱼ₂, fⱼ₃, fₖ₂, and fₖ₃ in FIG. 6), the descriptive data can be further transformed into a multi-dimensional space (e.g., 300-dimensional space) through sentence embedding. For example, the features fⱼ₂, fⱼ₃, fₖ₂, and fₖ₃ in FIG. 6 are transformed into sentence vectors Emⱼ₂, Emⱼ₃, Emₖ₂, and Emₖ₃, respectively. Thus, the pathology feature vectors Vⱼ and Vₖ can be transformed into vectors V′ⱼ and V′ₖ.

Each element of the vector V′ⱼ may be comparable to the corresponding element of the vector V′ₖ. Thus, the vectors V′ⱼ and V′ₖ can be comparable based on the comparisons between the corresponding elements of the vectors V′ⱼ and V′ₖ. That is, how the pathology reports 610 and 620 are similar can be determined based on the comparisons between the corresponding elements of the vectors V′ⱼ and V′ₖ.

For the category type data (e.g., Cⱼ₁, Cⱼ₅₀, Cₖ₁, and Cₖ₅₀) in the vectors V′ⱼ and V′ₖ, if one category of the vector V′ⱼ matches (or is identical to) the corresponding one of the vector V′ₖ, the comparison result between the two corresponding categories of the vectors V′ⱼ and V′ₖ would be 1 (e.g., indicating the match is true). If one category of the vector V′ⱼ does not match (or is not identical to) the corresponding one of the vector V′ₖ, the comparison result between the two corresponding categories of the vectors V′ⱼ and V′ₖ would be 0 (e.g., indicating the match is false). For example, if the category Cᵢⱼ of the vector V′ⱼ matches (or is identical to) the category Cₖ₁ of the vector V′ₖ, the comparison between the categories Cⱼ₁ and Cₖ₁ is 1 (e.g., indicating the match is true). If the category Cⱼ₅₀ of the vector V′ⱼ does not match (or is not identical to) the category Cₖ₅₀ of the vector V′ₖ, the comparison between the categories Cⱼ₅₀ and Cₖ₅₀ is 0 (e.g., indicating the match is false). For example, the comparison between the categories Cⱼ₁ and Cₖ₁ or between the categories Cⱼ₅₀ and Cₖ₅₀ may be represented as Equation (6).

$${match}_{n} = \{ \begin{matrix}1 , {C}_{jn} = {C}_{kn} \\ 0 , {C}_{jn} \neq {C}_{kn}\end{matrix} , 1 \leq n \leq 50 , n ∈ ℕ \qquad Equation ( 6 )$$

For the sentence vectors transformed though sentence embedding (e.g., Emⱼ₂, Emⱼ₃, Emₖ₂, and Emₖ₃) in the vectors V′ⱼ and V′ₖ, the similarity between two corresponding two-sentence vectors is determined based on their cosine similarity. For example, the similarity between the sentence vectors Emⱼ₂ and Emₖ₂ is determined based on the cosine similarity between the sentence vectors Emⱼ₂ and Emₖ₂. The similarity between the sentence vectors Emⱼ₃ and Emₖ₃ is determined based on the cosine similarity between the sentence vectors Emⱼ₃ and Emₖ₃. For example, the cosine similarity between the sentence vectors Emⱼ₂ and Emₖ₂ or between the sentence vectors Emⱼ₃ and Emₖ₃ may be represented as Equation (8). In Equation (8), the sentence vectors are in a 300-dimensional space.

$${similairty}_{n} = \frac{{Em}_{jn} \cdot {Em}_{kn}}{‖ {Em}_{jn} ‖ ‖ {Em}_{kn} ‖} = \frac{{\sum }_{i = 1}^{300} {Em}_{jni} \times {Em}_{kni}}{\sqrt{{\sum }_{i = 1}^{300} {( {Em}_{jni} )}^{2}} \times \sqrt{{\sum }_{i = 1}^{300} {( {Em}_{kni} )}^{2}}} , 1 \leq n \leq 50 , n ∈ ℕ \qquad Equation ( 7 )$$

The 50 weight values listed in Table 12 can be further applied to the similarity between two pathology reports. The normalization of the 50 weight values of Table 12 may be represented as Equations (9). The variables w₁ to w₅₀ indicate the 50 weight values listed in Table 12, and variables w′₁ to w′₅₀ indicate the corresponding 50 normalized weight values.

$${w}_{n}^{′} = \frac{{w}_{n}}{{\sum }_{r = 1}^{50} {w}_{r}} , 1 \leq n \leq 50 n ∈ ℕ \qquad Equation ( 8 )$$

The similarity score between the n-th feature of the pathology report and the n-th feature of the pathology report 620 can be the similarity score between the n-th feature of the vector V′ⱼ and the n-th feature of the vector V′ₖ. The similarity score of the n-th feature can be represented as Equation (9). When the n-th feature is numeric data or category type data, matchₙ would be by the corresponding normalized weight value. When the n-th feature is descriptive data or a sentence vector, similarityₙ would be multiplied by the corresponding normalized weight value.

$${score}_{jkn} = \{ \begin{matrix}{w}_{n}^{′} \cdot {match}_{n} , & where the n - th feature is numeric data \\ {w}_{n}^{′} \cdot {similarity}_{n} , & where the n - th feature is descriptive data\end{matrix} \qquad Equation ( 9 )$$

The similarity score between the pathology reports 610 and 620 can be the sum of the similarity scores of the first feature to the 50-th feature. The similarity score between the vectors V′ⱼ and V′ₖ can be the sum of the similarity scores of the first feature to the 50-th feature. The sum of the similarity scores of the first feature to the 50-th feature can be represented as Equation (10).

$${score}_{jkn} = {\sum }_{n = 1}^{50} {score}_{jkn} \qquad Equation ( 10 )$$

The present disclosure thus can provide an efficient way for clinicians to score the similarity between two cases. Since the similarity of two pathology reports of two patients can be scored, clinicians could search similar cases in the past more easily. Furthermore, the clinicians could know which parts of two cases are similar and why the two case are similar based on the scores for the 50 important pathological features as shown in Table 1.

Therefore, the present disclosure provides an interpretable method to score the similarity between two cases. In contrast, a lot of parameters and probabilities generated by a machine learning algorithm (or a deep learning algorithm) are not interpretable. The interpretability of the present disclosure makes clinicians able to explain where and why two cases are similar. The interpretability and similarity provided by the present disclose would be helpful for clinicians to evaluate subsequent diagnoses and therapies of a patient.

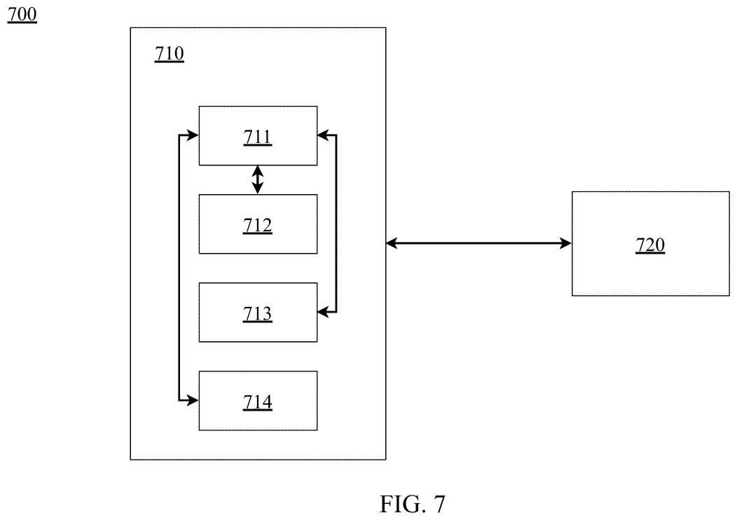

Referring to FIG. 7, it shows an example of a computer system 700 capable of performing one or more operations of the methods of the present disclosure. The computer system 700 includes, in at least some embodiments of the present disclosure, a computing device 710 and a database 720. The computing device 710 may be a server computer, a client computer, a personal computer (PC), a tablet PC, a set-top box (STB), a personal digital assistant (PDA), a cellular telephone, or a smartphone. The computing device 710 comprises processor 711, input/output interface 712, communication interface 713, and memory 714. The database 720 may store pathology reports from which key linguistic patterns would be extracted. The database 720 may store pathology reports to be analyzed or summarized. The input/output interface 712 is coupled with the processor 711. The input/output interface 712 allows the user to manipulate the computing device 710 in order to perform the operations or methods of the present disclosure (e.g., the method disclosed in FIG. 2). The communication interface 713 is coupled with the processor 711. The communication interface 713 allows the computing device 710 to communicate with the database 720. The communication interface 713 may support one or more of the following protocols: Universal Serial Bus (USB), Ethernet, Bluetooth, IEEE 802.11, 3GPP Long-Term Evolution (LTE) (4G), and 3GPP New Radio (5G). A memory 714 may be a non-transitory computer readable storage medium. The memory 714 is coupled with the processor 711. The memory 714 has stored program instructions that can be executed by one or more processors (for example, the processor 711). Upon execution of the program instructions stored on the memory 714, the program instructions cause performance of the one or more operations of the methods disclosed in the present disclosure. For example, the program instructions may cause the computing device 710 to perform a set of acts that at least include: (i) determining a confidence degree and a support degree between a linguistic term and a next linguistic term based on co-occurrences of the linguistic term and the next linguistic term; (ii) generating a set of candidate linguistic terms; (iii) generating a first set of linguistic patterns through performing random walks on the set of candidate linguistic terms; (iv) generating a second set of linguistic patterns through removing redundant linguistic patterns from the first set of linguistic patterns; and (v) outputting the key linguistic patterns based on the second set of linguistic patterns.

As another exemplary example, the program instructions may cause the computing device 710 to perform a method of summarizing a pathology report. The method may comprise acquiring a plurality of pathological features from the pathology report based on key linguistic patterns. The key linguistic patterns are generated according to any one the methods of the present disclosure.

The scope of the present disclosure is not intended to be limited to the particular embodiments of the process, machine, manufacture, and composition of matter, means, methods, steps, and operations described in the specification. As those skilled in the art will readily appreciate from the disclosure of the present disclosure, processes, machines, manufacture, composition of matter, means, methods, steps, or operations presently existing or later to be developed, that perform substantially the same function or achieve substantially the same result as the corresponding embodiments described herein may be utilized according to the present disclosure. Accordingly, the appended claims are intended to include within their scope, processes, machines, manufacture, and compositions of matter, means, methods, steps, or operations. In addition, each claim constitutes a separate embodiment, and the combination of various claims and embodiments are within the scope of the disclosure.

The methods, processes, or operations according to embodiments of the present disclosure can also be implemented on a programmed processor. However, the controllers, flowcharts, and modules may also be implemented on a general purpose or special purpose computer, a programmed microprocessor or microcontroller and peripheral integrated circuit elements, an integrated circuit, a hardware electronic or logic circuit such as a discrete element circuit, a programmable logic device, or the like. In general, any device on which resides a finite state machine capable of implementing the flowcharts shown in the figures may be used to implement the processor functions of present disclosure.

An alternative embodiment preferably implements the methods, processes, or operations according to embodiments of the present disclosure in a non-transitory, computer-readable storage medium storing computer programmable instructions. The instructions are preferably executed by computer-executable components preferably integrated with a network security system. The non-transitory, computer-readable storage medium may be stored on any suitable computer readable media such as RAMs, ROMs, flash memory, EEPROMs, optical storage devices (CD or DVD), hard drives, floppy drives, or any suitable device. The computer-executable component is preferably a processor, but the instructions may alternatively or additionally be executed by any suitable dedicated hardware device. For example, an embodiment of the present disclosure provides a non-transitory, computer-readable storage medium having computer programmable instructions stored therein.

While the present disclosure has been described with specific embodiments thereof, it is evident that many alternatives, modifications, and variations may be apparent to those skilled in the art. For example, various components of the embodiments may be interchanged, added, or substituted in the other embodiments. Also, all of the elements of each figure are not necessary for operation of the disclosed embodiments. For example, one of ordinary skill in the art of the disclosed embodiments would be enabled to make and use the teachings of the present disclosure by simply employing the elements of the independent claims. Accordingly, embodiments of the present disclosure as set forth herein are intended to be illustrative, not limiting. Various changes may be made without departing from the spirit and scope of the present disclosure.

Even though numerous characteristics and advantages of the present disclosure have been set forth in the foregoing description, together with details of the structure and function of the invention, the disclosure is illustrative only. Changes may be made in detail, especially in matters of shape, size, and arrangement of parts within the principles of the invention to the full extent indicated by the broad general meaning of the terms in which the appended claims are expressed.

What is claimed is:

1. A method of extracting key linguistic patterns from a pathology report, comprising:
determining a confidence degree and a support degree between a linguistic term and a next linguistic term based on co-occurrences of the linguistic term and the next linguistic term, wherein the linguistic term occurs prior to the next linguistic term in the pathology report;
generating a set of candidate linguistic terms, wherein the confidence degree between a candidate linguistic term and a corresponding next candidate linguistic term is equal to or greater than a confidence threshold, and the support degree between the candidate linguistic term and the corresponding next candidate linguistic term is equal to or greater than a support threshold;
generating a first set of linguistic patterns through performing random walks on the set of candidate linguistic terms; and
determining the key linguistic patterns through removing redundant linguistic patterns from the first set of linguistic patterns.

2. The method of claim 1, wherein:
the support degree between the linguistic term and the next linguistic term is generated based on the number of co-occurrences of the linguistic term and the next linguistic term; and
the confidence degree between the linguistic term and the next linguistic term is generated based on a probability of occurrence of the next linguistic term under the case that the linguistic term occurs.

3. The method of claim 1, wherein removing the redundant linguistic patterns from the first set of linguistic patterns includes removing a first linguistic pattern when the first linguistic pattern indicates a subset of a second linguistic pattern.

4. The method of claim 1, wherein the confidence degree between the linguistic term and the next linguistic term is irrelevant to a second confidence degree between a previous linguistic term and the linguistic term, wherein the previous linguistic term occurs prior to the linguistic term in the pathology report.

5. A method of summarizing a pathology report, comprising:
acquiring a plurality of pathological features from the pathology report based on key linguistic patterns, wherein the key linguistic patterns are generated according to the method of claim 1.

6. The method of claim 6, further comprising:
locating a description within a SOAP (subject, objective, assessment, and plan) section, wherein the description has four or more parts, and a “,” separates any two adjacent parts,
wherein when the description has four parts, a first part of the description is related to an organ, a second part of the description is related to a location, a third part of the description is related to a sampling method, and a four part of the description is related to a diagnosis.

7. The method of claim 6, wherein when the description has four parts, the method further comprising:
locating a first part related to an organ and a second part related to a sampling method based on the key linguistic patterns; and
locating a third part related to a location between the first and second parts;
locating a fourth part related to a diagnosis posterior to the second part.

8. The method of claim 7, wherein:
the first part related to the organ is located based a first subset of the key linguistic patterns, the first subset of the key linguistic patterns includes “lung,” “lymph node,” “brain,” and “skin”; and
the second part related to the sampling method is located based on a second subset of the key linguistic patterns, the second subset of the key linguistic patterns includes “biopsy,” “VATS,” and “EBUS.”

9. The method of claim 5, further comprising:
locating an immunohistochemistry (IHC) part based on a third subset of the key linguistic patterns,
wherein the third subset of the key linguistic patterns includes “immunohistochemical,” “immunohistochemically,” “immunohistochemical,” “immunohistochemically,” “immunostudy,” “IHC,” “immunostains,” “immunohistochemistry,” “immunostudy,” “immunoreactive,” “shows adenocarcinoma composed.”

10. The method of claim 9, further comprising:
locating a feature term of a fourth subset of the key linguistic patterns in the IHC part;
locating a first modifier term occurring prior to the feature term;
locating a second modifier term occurring posterior to the feature term; and
selecting one of the first and second modifier terms based on a first distance between the first modifier term and the feature term and a second distance between the second modifier term and the feature term,

11. The method of claim 10, wherein:
the fourth subset of the key linguistic patterns includes “CK7,” “TTF-1,” “Napsin A,” “CK20,” “P40,” “CDX2,” “P63,” “P16,” “cytokeratin (AE1/AE3),” “Vimentin,” “PAX-8,” “CD56,” “chromogranin-A,” “synaptophysin,” “GATA3,” “P53,” “S100,” “Ki67,” and “EBER”; and
the first and second modifier terms includes “positive” and “negative.”

12. The method of claim 6, further comprising:
locating at least one candidate segments based on a linguistic pattern of volume;
acquiring a volume in one of the at least one candidate segments as a tumor size when context of the one of the at least one candidate segments includes one key linguistic pattern of a fifth subset of the key linguistic patterns; and
determining a largest value of the tumor size as a greatest dimension,
wherein the fifth subset of the key linguistic patterns includes “on cut,” “firm tumor,” and “tumor measuring.”

13. The method of claim 12, further wherein the linguistic pattern of volume includes:
a first number, a second number, and a third number,
a multiplication symbol between the first and second numbers,
another multiplication symbol between the second and third numbers, and
a unit of length posterior to the third number.

14. The method of claim 5, further comprising:
locating a microscopic evaluation section base on a term of “microscopic evaluation”; and
locating a colon of an item of a first set of items in the microscopic evaluation section; and
acquiring information posterior to the colon as the item of the first set of items,
wherein the first set of items includes: tumor focality, histology type, histology grade, lymphovascular invasion, visceral pleura invasion, and closest margin.

15. The method of claim 5, further comprising:
locating a PD-L1 testing part base on a sixth subset of the key linguistic patterns; and
locating a colon of an item of a second set of items in the PD-L1 testing part; and
acquiring information posterior to the colon as the item of the second set of items,
wherein the sixth subset of the key linguistic patterns includes: “22C3,” “28-8,” “SP142,” and “SP263,” and
the second set of items includes: tumor proportion score (TPS), combined positive score (CPS), tumor cell (TC), and immune cells (IC).

16. The method of claim 5, further comprising:
locating an epidermal growth factor receptor (EGFR) part base on a term of “EGFR”; and
determining whether mutations are in exon 18, exon 19, exon 20, or exon 21 based on the terms of “18”, “19”, “20”, and “21”; and
determining a mutation is at position 790 of exon 20 based base on a term of “T790M.”

17. The method of claim 5, further comprising:
locating a molecular test part base on a seventh subset of key linguistic patterns; and
identifying a modifier term in context of one key linguistic pattern of the seventh subset of key linguistic patterns; and
determining a mutation is in a gene related to the one key linguistic pattern when the modifier term is identified as “positive,”
wherein the seventh subset of the key linguistic patterns includes: “ALK,” “ROS1,” “BRAF,” “MET,” “KRAS,” “ERBB2,” “PIK3CA,” “NRAS,” “MEK1,” “NTRK,” and “RET.”

18. The method of claim 5, further comprising:
locating a pathologic staging (pTNM) part based on a term of “pathologic staging” and a term of “pTNM”; and
retrieving a first stage indicator based on a term of “pT”;
retrieving a second stage indicator based on a term of “pN”; and
retrieving a third stage indicator based on a term of “pM.”

19. The method of claim 5, further comprising:
transforming the pathology report into a first vector in terms of the plurality of pathological features, wherein the first vector includes multiple elements of category and multiple elements of sentence vector;
transforming a second pathology report into a second vector in terms of the plurality of pathological features wherein the second vector includes multiple elements of category and multiple elements of sentence vector; and
calculating a similarity score between the pathology report and the second pathology report through summing each score of corresponding elements of the first vector and the second vector,
wherein, when a n-th element is an element of category, a score of the n-th element is
$${score}_{n} = \{ \begin{matrix}{w}_{n} , {C}_{1 n} = {C}_{2 n} \\ 0 , {C}_{1 n} \neq {C}_{2 n}\end{matrix} ,$$
C₁ₙ indicates the n-th element of the first vector, C₂ₙ indicates the n-th element of the second vector, wₙ indicates a weight value for the n-th element, and
wherein, when the n-th element is an element of sentence vector, the score of the n-th element is
$${score}_{n} = {w}_{n} \times \frac{{Em}_{1 n} \cdot {Em}_{2 n}}{‖ {Em}_{1 n} ‖ ‖ {Em}_{2 n} ‖} ,$$
Em₁ₙ indicates the n-th element of the first vector, Em₂ₙ indicates the n-th element of the second vector.

20. A non-transitory computer storage medium having stored thereon program instructions that, upon execution by a processor, cause performance of a set of operations according to the method of claim 1.
